(19) United States
(12) Patent Application Publication
(10) Pub. No.: US 2025/0383533 A1
(43) Pub. Date: Dec. 18, 2025

(54) SURGICAL MICROSCOPE SYSTEM, AUXILIARY IMAGING DEVICE, AND SURGICAL FIELD IMAGING METHOD

(71) Applicant: TEIKYO UNIVERSITY, Tokyo (JP)

(72) Inventor: Masayuki Okochi, Tokyo (JP)

(21) Appl. No.: 18/877,741

(22) PCT Filed: May 31, 2023
(86) PCT No.: PCT/JP2023/020264

§ 371 Date: Dec. 20, 2024

(30) Foreign Application Priority Data
Jul. 8, 2022 (JP) 2022-110368

(51) Int. Cl. G02B 21/00; A61B 17/29; A61B 90/00; A61B 90/30; G02B 21/02

(52) U.S. Cl. CPC G02B 21/0012; A61B 17/29; A61B 90/30; A61B 90/361; G02B 21/02

(57) ABSTRACT

In a surgical microscope system including: a surgical microscope main body having an observation optical system including at least an objective lens; and an auxiliary imaging device having at least an imaging unit and a lighting unit, the auxiliary imaging device images a surgical field in the state of being connected to a surgical instrument held by a surgeon or a surgical assistant.

### TECHNICAL FIELD

The present invention relates to a surgical microscope system, an auxiliary imaging device, and a surgical field imaging method.

Priority is claimed on Japanese Patent Application No. 2022-110368, filed Jul. 8, 2022, the content of which is incorporated herein by reference.

### BACKGROUND ART

In Patent Document 1, a surgical microscope system has been described. In the surgical microscope system described in Patent Document 1, a microscope device and a display device are included. In the technology described in Patent Document 1, the microscope device has a microscope unit that images an observation target and outputs a video signal, a support part that is configured as a balance arm supporting the microscope unit, and an auxiliary observation device that can be attached/detached to/from the support part and is configured to be able to observe an observation range according to the microscope unit. In addition, in the technology described in Patent Document 1, the auxiliary observation device has an eyepiece unit that a surgeon looks into.

However, in the technology described in Patent Document 1, the auxiliary observation device is, for example, a loupe with an eyepiece unit and is disposed at a position quite far from a surgical part. For this reason, in accordance with the technology described in Patent Document 1, for example, an image that is actually desired by a surgeon such as a imaged image of the surgical field from the position of a surgical instrument held by the surgeon or a surgical assistant in microsurgery cannot be provided for the surgeon.

### CITATION LIST

### Patent Document

Patent Document 1: U.S. Pat. No. 6,656,237

### SUMMARY OF INVENTION

### Technical Problem

In consideration of the points described above, an object of the present invention is to provide a surgical microscope system, an auxiliary imaging device, and a surgical field imaging method capable of providing a imaged image of the surgical field from the position of a surgical instrument held by a surgeon or a surgical assistant in microsurgery, for the surgeon.

### Solution to Problem

According to one aspect of the present invention, there is provided a surgical microscope system including: a surgical microscope main body having an observation optical system including at least an objective lens; and an auxiliary imaging device including at least an imaging unit and a lighting unit, wherein the auxiliary imaging device images a surgical field in the state of being connected to a surgical instrument held by a surgeon or a surgical assistant and with an angle different from that of the surgical microscope main body.

According to one aspect of the present invention, there is provided an auxiliary imaging device included in a surgical microscope system including a surgical microscope main body having an observation optical system including at least an objective lens, the auxiliary imaging device including: at least an imaging unit and a lighting unit; wherein a surgical field is imaged in the state of being connected to a surgical instrument held by a surgeon or a surgical assistant and with an angle different from that of the surgical microscope main body.

According to one aspect of the present invention, there is provided a surgical field imaging method including: a first imaging step of imaging a surgical field by using a surgical microscope main body having an observation optical system including at least an objective lens; and a second imaging step of imaging the surgical field with an angle different from that of the surgical microscope main body by using an auxiliary imaging device having at least an imaging unit and a lighting unit, wherein the auxiliary imaging device images the surgical field in the state of being connected to a surgical instrument held by a surgeon or a surgical assistant.

### Advantageous Effects of Invention

According to the present invention, a surgical microscope system, an auxiliary imaging device, and a surgical field imaging method capable of providing a imaged image of the surgical field from the position of a surgical instrument held by a surgeon or a surgical assistant in microsurgery, for the surgeon can be provided.

### BRIEF DESCRIPTION OF DRAWINGS

FIG. 1 A diagram illustrating one example of a surgical microscope system 1 according to a first embodiment.

FIG. 2 A diagram conceptually illustrating microvascular anastomosis in microsurgery performed using the surgical microscope system 1 according to the first embodiment.

FIG. 3 A diagram illustrating one example of forceps ST1 and a connecting member 13 in a state in which the connecting member 13 is attached to the forceps ST1.

FIG. 4 A diagram illustrating one example of the forceps ST1 and the connecting member 13 in the state in which the connecting member 13 is attached to the forceps ST1.

FIG. 5 A diagram illustrating one example of a housing unit 12C of an auxiliary imaging device 12, a connecting member 13, and forceps ST1 in a state in which the housing unit 12C of the auxiliary imaging device 12, the connecting member 13, and the forceps ST1 are connected.

FIG. 6 A diagram simulating an appearance in which the forceps ST1 among the housing unit 12C of the auxiliary imaging device 12, the connecting member 13, and the forceps ST1 in a state in which the housing unit 12C of the auxiliary imaging device 12, the connecting member 13, and the forceps ST1 are connected is held by a surgeon and is used for microvascular anastomosis in microsurgery.

FIG. 7 A diagram simulating an appearance in which the forceps ST1 among the housing unit 12C of the auxiliary imaging device 12, the connecting member 13, and the forceps ST1 in a state in which the housing unit 12C of the auxiliary imaging device 12, the connecting member 13, and the forceps ST1 are connected is held by a surgeon and is used for microvascular anastomosis in microsurgery.

FIG. 8 A diagram simulating an appearance in which the forceps ST1 among the housing unit 12C of the auxiliary imaging device 12, the connecting member 13, and the forceps ST1 in a state in which the housing unit 12C of the auxiliary imaging device 12, the connecting member 13, and the forceps ST1 are connected is held by a surgeon and is used for microvascular anastomosis in microsurgery.

FIG. 9 A diagram in which one example of an image of a surgical field imaged by a surgical microscope main body 11 and one example of an image of a surgical field imaged by the auxiliary imaging device 12 are compared with each other.

FIG. 10 A diagram for describing information of micro blood vessels BL that are necessary for a surgeon when microvascular anastomosis is performed in microsurgery.

FIG. 11 A sequence diagram for describing one example of a process executed by the surgical microscope system 1 according to the first embodiment.

FIG. 12 A diagram illustrating one example of a surgical microscope system 1 according to a second embodiment.

### DESCRIPTION OF EMBODIMENTS

Hereinafter, embodiments of a surgical microscope system, an auxiliary imaging device, and a surgical field imaging method of the present invention will be described with reference to the drawings.

### First Embodiment

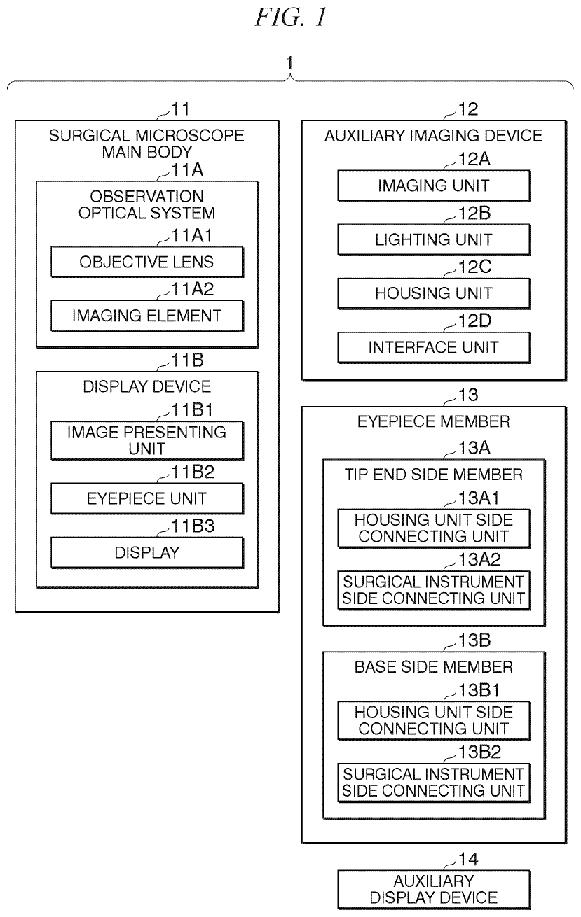

FIG. 1 is a diagram illustrating one example of a surgical microscope system 1 according to a first embodiment.

In the example illustrated in FIG. 1, the surgical microscope system 1 according to the first embodiment, for example, is used when a reconstructive surgery for microvascular anastomosis is performed in microsurgery.

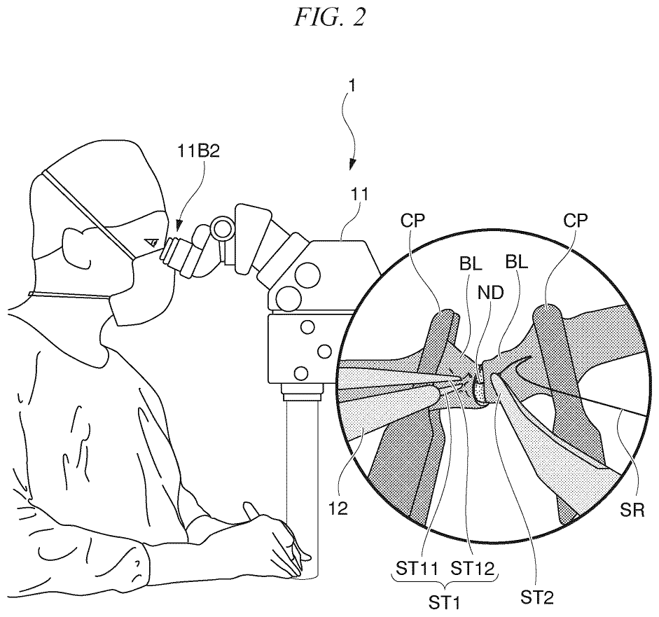

FIG. 2 is a diagram conceptually illustrating microvascular anastomosis in microsurgery performed using the surgical microscope system 1 according to the first embodiment. In more detail, a part enclosed by a circle in FIG. 2 represents a surgical part included in a surgical field that is imaged by a surgical microscope main body 11 illustrated in FIGS. 1 and 2 (two micro blood vessels BL to be anastomosed in micro surgery and the like).

In the example illustrated in FIGS. 1 and 2, the surgical microscope system 1 according to the first embodiment includes a surgical microscope main body 11, an auxiliary imaging device 12, a connecting member 13, and an auxiliary display device 14.

The surgical microscope main body 1 according to the first embodiment, for example, is configured to be similar to a surgical microscope described on websites indicated by the following URLs.

```
  https://www.leica-
microsystems.com/jp/%E8%A3%BD%E5%93%81%E7%B4%B9%E4%BB%8B/%E6%
89%8B%E8%A1%93%E7%94%A8%E9%A1%95%E5%BE%AE%E9%8F%A1/
  https://www.medicaltown.net/neurosurgery/product/sms/orbeye/
  https://products.awi.co.jp/ja/medical/medical/equipment/id000172
```

In the example illustrated in FIGS. 1 and 2, the surgical microscope main body 11 has an observation optical system 11A and a display device 11B. The observation optical system 11A, for example, has functions for acquiring an image of a surgical field, changing the magnification ratio, matching the focus, and the like. The observation optical system 11A, for example, includes an objective lens 11A1, an imaging element 11A2, and the like. The objective lens 11A1 is disposed to face a surgical field including surgical parts such as two micro blood vessels BL (see FIG. 2) to be anastomosed in microsurgery. The imaging element 11A2 converts light of a surgical field entering the observation optical system 11A through the objective lens 11A1 into digital image data and outputs the digital image data. In other words, the imaging element 11A2 images a surgical field through the objective lens 11A1. In other words, the observation optical system 11A projects a surgical field using an optical system such as the objective lens 11A1 or the like and achieves the role of guiding a projection image of the surgical field to the imaging element 11A2.

The display device 11B has a function of displaying an image of a surgical field corresponding to the digital image data of the surgical field generated by the imaging element 11A2. The display device 11B, for example, includes an image presenting unit 11B1, an eyepiece unit 11B2, a display 11B3, and the like. The image presenting unit 11B1 presents an image of a surgical field corresponding to digital image data of the surgical field generated by the imaging element 11A2 to the eyes of a surgeon through the eyepiece unit 11B2. The display 11133 displays an image of a surgical field corresponding to digital image data of the surgical field generated by the imaging element 11A2. A surgeon can check an image of a surgical field through the eyepiece unit 11B2 and also can check the image of the surgical field using the display 11B3. The display 11B3 is used also for a surgical assistant, an instrument nurse, and the like to check an image of a surgical field.

In the example illustrated in FIG. 1, although the surgical microscope main body 11 includes the display 11B3, in another example, the surgical microscope main body 11 may not include the display 11B3.

In the example illustrated in FIGS. 1 and 2, the auxiliary imaging device 12, for example, has an imaging unit 12A, a lighting unit 12B, a housing unit 12C, an interface unit 12D, and the like.

The imaging unit 12A images surgical parts (two micro vessels BL or the like to be anastomosed in microsurgery) included in a surgical field imaged using the surgical microscope main body 11 with an angle different from that of the surgical microscope main body 11. The lighting unit 12B emits illumination light to a surgical part imaged by the imaging unit 12A. The housing unit 12C houses the imaging unit 12A and the lighting unit 12B.

The interface unit 12D has a function of transmitting image data of surgical parts imaged by the imaging unit 12A to the auxiliary display device 14. The interface unit 12D, for example, is configured using a Universal Serial Bus (USB) cable and the like. The auxiliary display device 14 displays an image of surgical parts (two micro blood vessels BL to be anastomosed in microsurgery) imaged using the imaging unit 12A with an angle different from that of the surgical microscope main body 11.

In the example illustrated in FIGS. 1 and 2, although image data of surgical parts imaged by the imaging unit 12A is transmitted to the auxiliary display device 14 by the interface unit 12D configured using a USB cable and the like, in another example, image data of surgical parts image by the imaging unit 12A may be transmitted to the auxiliary display device 14 using the interface unit 12D having a radio communication function.

In the example illustrated in FIGS. 1 and 2, a surgeon can check a surgical field through the eyepiece unit 11B2 as described above and also can check surgical parts (two micro blood vessels BL to be anastomosed in microsurgery) with an angle different from that of the surgical microscope main body 11 by using the auxiliary display device 14.

The auxiliary imaging device 12, for example, is configured similar to an ear scope, an ear pick camera, or the like described on web sites indicated by URLs illustrated below.

```
  https://www.amazon.co.jp/dp/B08Y8893B2 tag=sl01tn-
22&linkCode=osi&th=1&psc=1https://www.eselect-ec.jp/category/select/pid/9190
  https://buy.sympheia.shop/bellarmine/jdMcJBemeE.html
  https://www.biccamera.com/bc/item/5915548/
```

The connecting member 13 connects a surgical instrument ST held by a surgeon and the auxiliary imaging device 12.

As illustrated in FIG. 2, when microvascular anastomosis is performed in microsurgery, a needle holder ST2 as a surgical instrument ST is held by a surgeon's dominant hand. The needle holder ST2 grips a suture needle ND to which a suture thread SR is connected.

In addition, when microvascular anastomosis is performed in microsurgery, forceps ST1 as a surgical instrument ST are held by a surgeon's non-dominant hand. The forceps ST1 are fixed by a clip CP and performs gripping of one micro blood vessel BL out of two micro blood vessels BL that are sutured using the suture thread SR and the like.

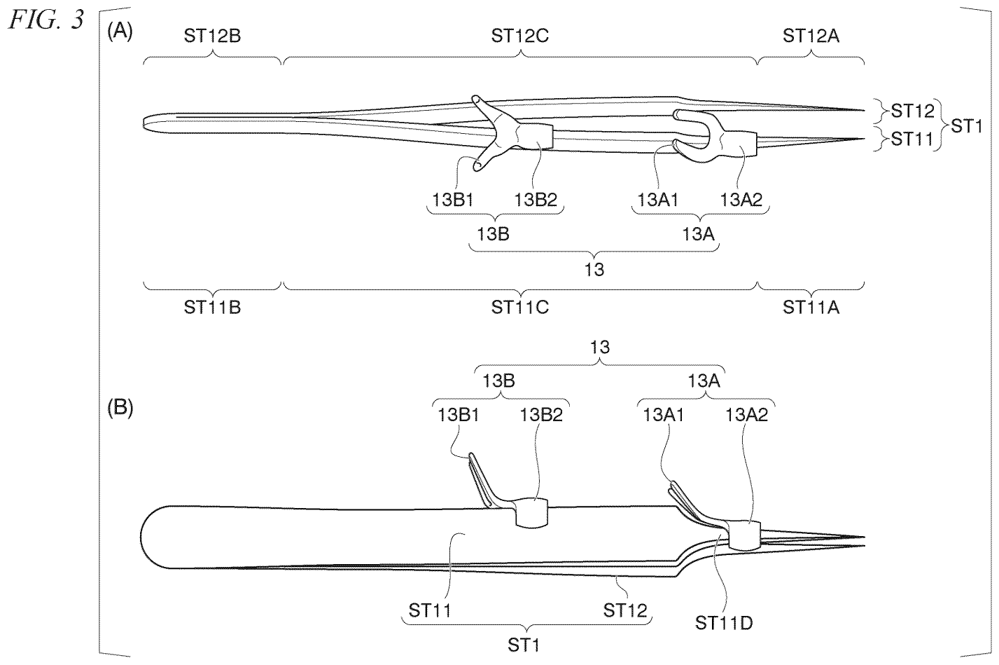

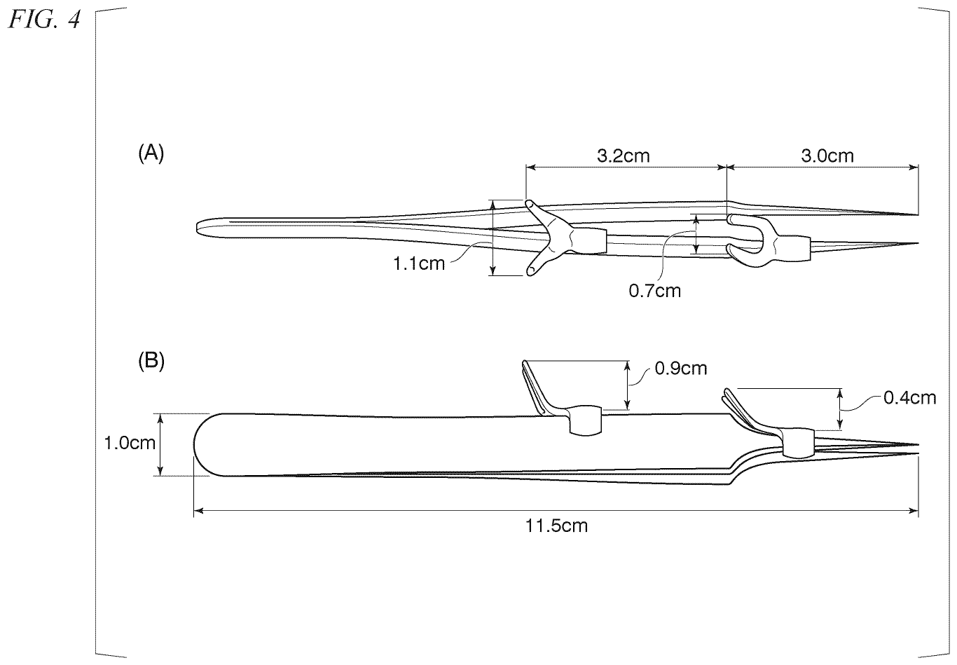

FIGS. 3 and 4 are diagrams illustrating one example of forceps ST1 and a connecting member 13 in a state in which the connecting member 13 is attached to the forceps ST1. In more detail, FIGS. 3(A) and 4(A) correspond to drawings of forceps ST1 and a connecting member 13 seen from an upper side of the forceps ST1 and the connecting member 13 (in other words, from an objective lens 11A1 of a surgical microscope main body 11), and FIGS. 3(B) and 4(B) correspond to diagrams of the forceps ST1 and the connecting member 13 seen from a lower side of FIGS. 3(A) and 4(A). FIG. 3 is a diagram for describing one example of parts of the forceps ST1 and the connecting member 13, and FIG. 4 is a diagram for describing one example of dimensions of the forceps ST1 and the connecting member 13.

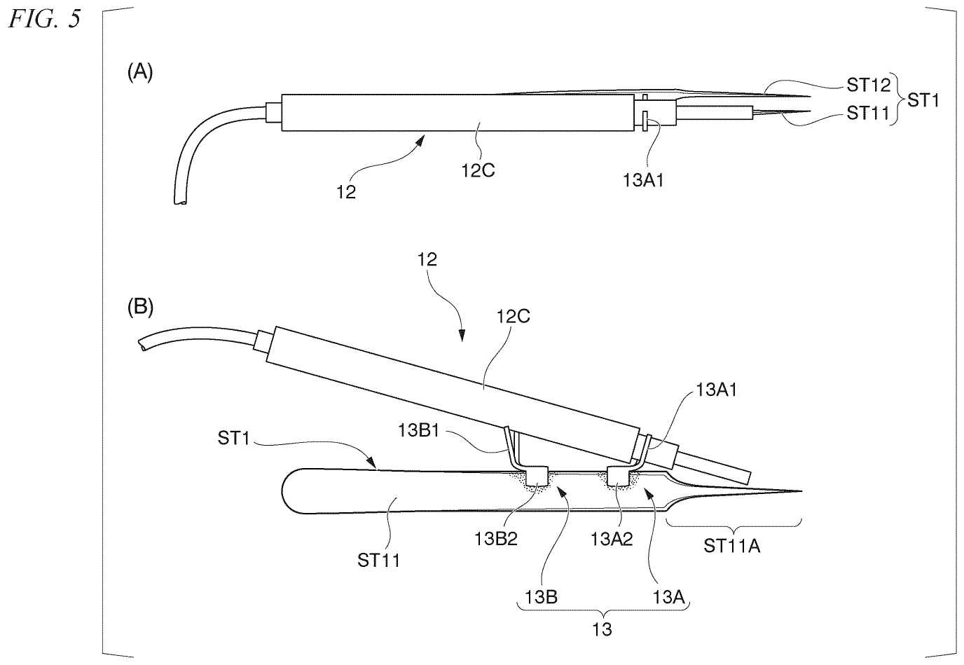

FIG. 5 is a diagram illustrating one example of a housing unit 12C of an auxiliary imaging device 12, a connecting member 13, and forceps ST1 in a state in which the housing unit 12C of the auxiliary imaging device 12, the connecting member 13, and the forceps ST1 are connected. In more detail, FIG. 5(A) corresponds to a diagram of the housing unit 12C of the auxiliary imaging device 12, the connecting member 13, and the forceps ST1 seen from an upper side of the housing unit 12C of the auxiliary imaging device 12, the connecting member 13, and the forceps ST1 (in other words, from the objective lens 11A1 of the surgical microscope main body 11), and FIG. 5(B) corresponds to a diagram of the housing unit 12C of the auxiliary imaging device 12, the connecting member 13, and the forceps ST1 seen from a lower side of FIG. 5(A).

In the example illustrated in FIGS. 1 to 5, a first part ST11 and a second part ST12 are included in the forceps ST1. The first part ST11 is a part that corresponds to the thumb of a surgeon's non-dominant hand (in other words, a hand on a side not holding the needle holder ST2 out of both hands of the surgeon). The second part ST12 is a part that corresponds to the index finger of a surgeon's non-dominant hand. In other words, in the example illustrated in FIGS. 1 to 5, the surgeon's right hand is the dominant hand, and the surgeon's left hand is the non-dominant hand.

In the example illustrated in FIGS. 3 and 4, a tip end part ST11A, a base part ST11B, a holding part ST11C, a first attachment part ST11D, and a second attachment part ST11E are included in the first part ST11. The tip end part ST11A is a part that grips a micro blood vessel BL and the like. The base part ST11B, for example, is a part that is bonded to the second part ST12 of the forceps ST1 using welding or the like.

In other words, in the example illustrated in FIGS. 3 and 4, the forceps ST1 are forceps of so-called “combined-type” that are configured by joining two members together through welding or the like.

In another example, the forceps ST1 may be so-called forceps of “bent-type” that are configured by bending one member.

In the example illustrated in FIGS. 3 and 4, the holding part ST11C is a part between the tip end part ST11A and the base part ST11B and is a part held by a surgeon. In more detail, the holding part ST11C is a part that is brought into contact with the thumb of a surgeon's non-dominant hand. The first attachment part ST11D is a part to which a tip end side member 13A, which will be described below, of the connecting member 13 is attached. The second attachment part ST11E is a part to which a base side member 13B, which will be described below, of the connecting member 13 is attached.

In the second part ST12, a tip end part ST12A, a base part ST12B, and a holding part ST12C are included. The tip end part ST12A is a part that grips micro blood vessels BL in cooperation with the tip end part ST11A of the first part ST11. The base part ST12B, for example, is a part that is jointed to the base part ST11B of the first part ST11 using welding or the like. The holding part ST12C is a part between the tip end part ST12A and the base part ST12B and is apart held by a surgeon. In more detail, the holding part ST12C is a part that is brought into contact with an index finger of the surgeon's non-dominant hand.

In addition, in the example illustrated in FIGS. 3 and 4, the tip end side member 13A and the base side member 13B are included in the connecting member 13. The tip end side member 13A is a member that is disposed on the tip end side of the forceps ST1 (the right side in FIGS. 3 and 4) out of the tip end side member 13A and the base side member 13B. The base side member 13B is a member disposed on a base side (the right side in FIGS. 3 and 4) of the forceps ST1 relative to the tip end side member 13A.

The tip end side member 13A includes a housing unit side connecting unit 13A1 and a surgical instrument side connecting unit 13A2.

The surgical instrument side connecting unit 13A2 is a part that is attached to the first attachment part ST11D of the first part ST11 of the forceps ST1. As illustrated in FIGS. 3 and 4, the surgical instrument side connecting unit 13A2 is configured by bending a part of a plate-shaped member configuring the tip end side member 13A.

In the example illustrated in FIGS. 3 and 4, by crimping the surgical instrument side connecting unit 13A2 onto the first attachment part ST11D of the first part ST11 of the forceps ST1, the surgical instrument side connecting unit 13A2 of the tip end side member 13A and the first part ST11 of the forceps ST1 are connected.

In another example, the surgical instrument side connecting unit 13A2 of the tip end side member 13A may have a shape different from the example illustrated in FIGS. 3 and 4, and the tip end side member 13A and the forceps ST1 may be connected using a technique (for example, adhesion, bonding, welding, press-fitting, or the like) different from the example illustrated in FIGS. 3 and 4.

In the example illustrated in FIGS. 3 and 4, the housing unit side connecting unit 13A1 is a part that is connected to the housing unit 12C of the auxiliary imaging device 12. As illustrated in FIGS. 3, 4, and 5, the housing unit side connecting unit 13A1 has an approximately “U” shape and a complementary shape to an approximately cylindrical outer surface of the housing unit 12C of the auxiliary imaging device 12.

In the example illustrated in FIGS. 3, 4, and 5, the housing unit 12C of the auxiliary imaging device 12 and the tip end side member 13A are connected to each other by press-fitting the housing unit 12C of the auxiliary imaging device 12 to the housing unit side connecting unit 13A1 of the tip end side member 13A.

In another example, the housing unit side connecting unit 13A1 of the tip end side member 13A may have a shape different from the example illustrated in FIGS. 3, 4, and 5, and the tip end side member 13A and the housing unit 12C of the auxiliary imaging device 12 may be connected to each other using a technique different from the example illustrated in FIGS. 3, 4, and 5 (for example, adhesion, bonding, welding, crimping, or the like).

In the example illustrated in FIGS. 3 and 4, the base side member 13B includes a housing unit side connecting unit 13B1 and a surgical instrument side connecting unit 13B2.

The surgical instrument side connecting unit 13B2 is a part that is attached to the second attachment part STIIE of the first part ST11 of the forceps ST1. As illustrated in FIGS. 3 and 4, the surgical instrument side connecting unit 13B2 is configured by bending a part of a plate-shaped member configuring the base side member 13B.

In the example illustrated in FIGS. 3 and 4, by crimping the surgical instrument side connecting unit 13B2 onto the second attachment part ST11E of the first part ST11 of the forceps ST1, the surgical instrument side connecting unit 13B2 of the base side member 13B and the first part ST11 of the forceps ST1 are connected.

In another example, the surgical instrument side connecting unit 13B2 of the base side member 13B may have a shape different from the example illustrated in FIGS. 3 and 4, and the base side member 13B and the forceps ST1 may be connected using a technique (for example, adhesion, bonding, welding, press-fitting, or the like) different from the example illustrated in FIGS. 3 and 4.

In the example illustrated in FIGS. 3 and 4, the housing unit side connecting unit 13B1 is a part that is connected to the housing unit 12C of the auxiliary imaging device 12. As illustrated in FIGS. 3, 4, and 5, the housing unit side connecting unit 13A1 has an approximate “U” shape.

In the example illustrated in FIGS. 3, 4, and 5, as described above, by press-fitting the housing unit 12C of the auxiliary imaging device 12 into the housing unit side connecting unit 13A1 of the tip end side member 13A, the housing unit 12C of the auxiliary imaging device 12 is brought into contact with (in other words, connected to) the housing unit side connecting unit 13B1 of the base side member 13B.

In another example, the housing unit side connecting unit 13111 of the base side member 13B may have a shape different from the example illustrated in FIGS. 3, 4, and 5, and, different from the example illustrated in FIGS. 3, 4, and 5 in which the housing unit 12C of the auxiliary imaging device 12 is brought into contact with the housing unit side connecting unit 13B1 of the base side member 13B, for example, by using adhesion, bonding, welding, press-fitting, crimping, or the like, the housing unit 12C of the auxiliary imaging device 12 and the housing unit side connecting unit 13B1 of the base side member 13B may be combined (fixed).

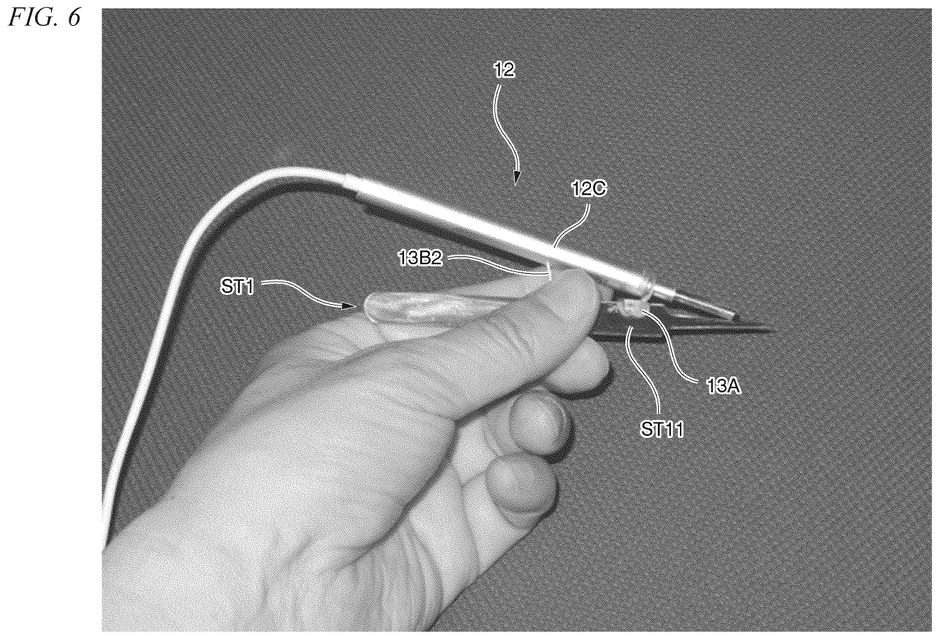

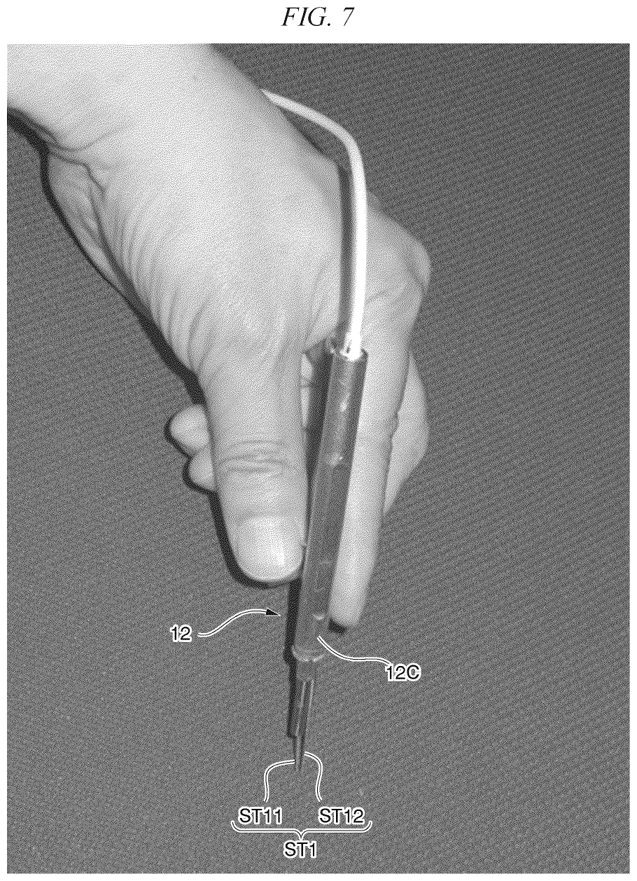

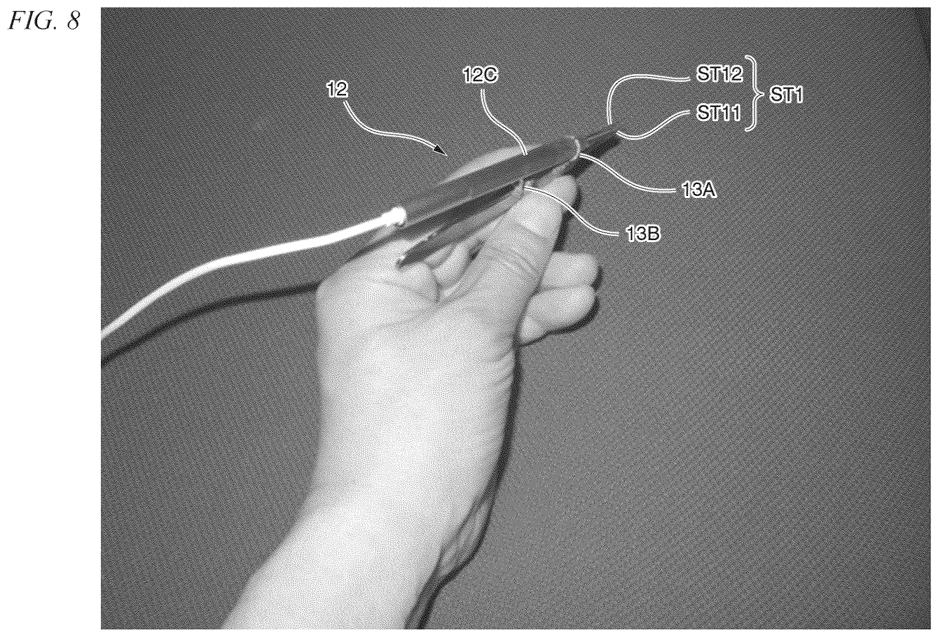

FIGS. 6 to 8 are diagrams simulating an appearance in which the housing unit 12C of the auxiliary imaging device 12 and the forceps ST1 out of the connecting member 13 and the forceps ST1 in a state in which the housing unit 12C of the auxiliary imaging device 12, the connecting member 13, and the forceps ST1 are connected is held by a surgeon and is used in microvascular anastomosis in microsurgery.

In the example illustrated in FIGS. 6 to 8, the auxiliary imaging device 12 images a surgical field with an angle different from that of the surgical microscope main body 11 in the state of being connected to the forceps ST1 as a surgical instrument ST held by a surgeon.

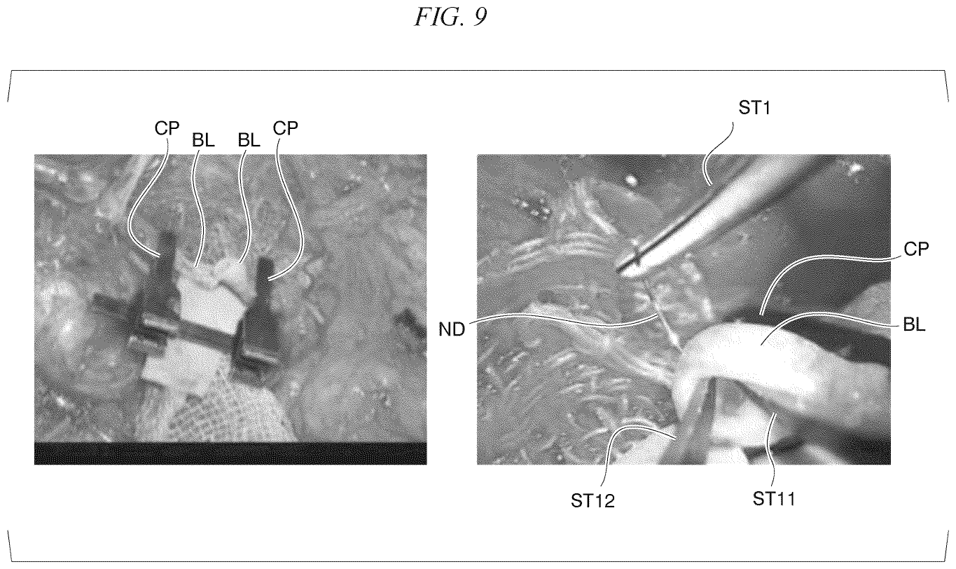

FIG. 9 is a diagram in which one example of an image of a surgical field imaged by the surgical microscope main body 11 and one example of an image of a surgical field imaged by the auxiliary imaging device 12 are compared with each other. In more detail, FIG. 9(A) illustrates one example of an image of a surgical field imaged by the surgical microscope main body 11, and FIG. 9(B) illustrates one example of an image of a surgical field imaged by the auxiliary imaging device 12.

As illustrated in FIG. 9(A), a center axis line of two micro blood vessels BL anastomosed in microsurgery extends in a direction (a horizontal direction in FIG. 9(A)) that is approximately vertical to the optical axis of the objective lens 11A1 of the observation optical system 11A of the surgical microscope main body 11. For this reason, a surgeon cannot perceive the inside of the micro blood vessels BL to be anastomosed in microsurgery in details by using only an image of a surgical field imaged by the surgical microscope main body 11 illustrated in FIG. 9(A).

As illustrated in FIG. 9(B), the center axis line of two micro blood vessels BL anastomosed in microsurgery forms a small acute angle (for example, an acute angle smaller than 30°) with the optical axis of the imaging unit 12A of the auxiliary imaging device 12. For this reason, as illustrated in FIG. 9(B), by using an image of a surgical field imaged by the auxiliary imaging device 12 (in other words, an image of the surgical field illustrated in FIG. 9(B)) in a state in which an end portion of a micro blood vessel BL facing the imaging unit 12A of the auxiliary imaging device 12 (the micro blood vessel BL on the right side of FIG. 9(A) out of two micro blood vessels BL illustrated in FIG. 9(A)) out of two micro blood vessels BL to be anastomosed in microsurgery is broadened by the first part ST11 and the second part ST12 of the forceps ST1, a surgeon can perceive the inside of the micro blood vessel BL (the micro blood vessel BL illustrated in FIG. 9(B)) facing the imaging unit 12A of the auxiliary imaging device 12 in detail.

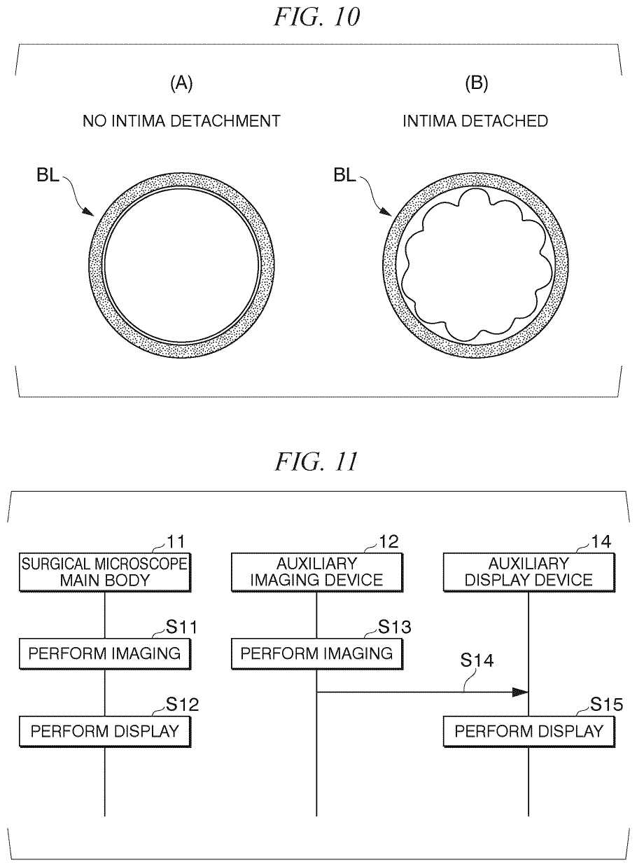

FIG. 10 is a diagram for describing information of micro blood vessels BL that are necessary for a surgeon when microvascular anastomosis is performed in microsurgery. In more detail, FIG. 10(A) illustrates a cross-section of a micro blood vessel BL of which an intima is not detached, and FIG. 10(B) illustrates a cross-section of a micro blood vessel BL of which an intima is detached.

As illustrated in FIG. 10(B), in a case in which an intima of an end portion (in other words, a part of the micro blood vessel BL that is sutured by a suture thread SR) of a micro blood vessel BL to be anastomosed in microsurgery is detached, a surgeon needs to take a measure to prevent thrombus formation in the micro blood vessel BL caused by the detached intima after the micro blood vessels are anastomosed in microsurgery (more specifically, a treatment pressing a detached intima to the inner wall of the micro blood vessel BL using the suture thread SR). In order to determine whether or not such a treatment needs to be performed, a surgeon needs information indicating whether or not the intima of the end portion of a micro blood vessel BL to be anastomosed in microsurgery is detached.

Meanwhile, as described above, since the center axis line of two micro blood vessels BL to be anastomosed in microsurgery extends in a direction (a horizontal direction in FIG. 9(A)) that is approximately vertical to the optical axis of the objective lens 11A1 of the observation optical system 11A of the surgical microscope main body 11, by using only an image of a surgical field imaged by the surgical microscope main body 11 illustrated in FIG. 9(A), a surgeon cannot perceive the inside of the end portion of a micro blood vessel BL to be anastomosed in microsurgery in detail and, it is difficult for the surgeon to acquire information indicating whether or not the intima of the end portion of the micro blood vessel BL is detached.

Thus, in the surgical microscope system 1 according to the first embodiment, as illustrated in FIG. 9(B), the imaging unit 12A of the auxiliary imaging device 12 images a surgical field from which the intima of the inside of the end portion of the micro blood vessel BL to be anastomosed in microsurgery can be checked. For this reason, according to the surgical microscope system 1 of the first embodiment, an image of the surgical field illustrated in FIG. 9(B), from which the intima of the inside of the end portion of the micro blood vessel BL to be anastomosed in microsurgery can be checked, seen from the position of the forceps ST1 can be provided for a surgeon.

Furthermore, in the surgical microscope system 1 according to the first embodiment, as illustrated in FIGS. 6 to 8, the surgical instrument side connecting unit 13A2 of the tip end side member 13A and the surgical instrument side connecting unit 13B2 of the base side member 13B are connected to the first part ST11, which is brought into contact with the thumb of the surgeon's non-dominant hand, out of the first part ST11 and the second part ST12 of the forceps ST1. In other words, the imaging unit 12A of the auxiliary imaging device 12 is connected to the first part ST11, which is brought into contact with the thumb of the surgeon's non-dominant hand, out of the first part ST11 and the second part ST12 of the forceps ST1 through the connecting member 13.

For this reason, according to the surgical microscope system 1 of the first embodiment, when moving the tip end part ST12A of the second part ST12 of the forceps ST1 by moving an index finger to the thumb side, a surgeon can suppress the shaking of the image of the surgical field imaged by the imaging unit 12A.

FIG. 11 is a sequence diagram for describing one example of a process executed by the surgical microscope system 1 according to the first embodiment.

In the example illustrated in FIG. 11, in Step S11, the observation optical system 11A of the surgical microscope main body 11 images the surgical field as illustrated in FIG. 9(A).

Next, in Step S12, the display device 11B of the surgical microscope main body 11 displays the image of the surgical field imaged in Step S11.

In the example illustrated in FIG. 11, in parallel with Steps S11 and S12, Steps S13 to S15 are executed.

In more detail, in Step S13, the imaging unit 12A of the auxiliary imaging device 12 images the surgical field as illustrated in FIG. 9(B) in a state in which the auxiliary imaging device 12 is connected to the forceps ST1 held by a surgeon. In other words, the imaging unit 12A of the auxiliary imaging device 12 images the surgical field with an angle different from the angle as illustrated in FIG. 9(A).

Next, in Step S14, the interface unit 12D of the auxiliary imaging device 12 transmits image data of the surgical field imaged in Step S13 to the auxiliary display device 14.

Next, in Step S15, the auxiliary display device 14 displays an image of the surgical field imaged in Step S13.

The surgeon performs microvascular anastomosis in microsurgery by selectively using the image of the surgical field displayed in Step S12 and the image of the surgical field displayed in Step S15.

As described above, in the surgical microscope system 1 according to the first embodiment, the auxiliary imaging device 12 images the surgical field with an angle different from that of the surgical microscope main body 11 in the state of being connected to the forceps ST1 held by the surgeon.

For this reason, in the surgical microscope system 1 according to the first embodiment, a imaged image of a surgical field from the position of forceps ST1 held by a surgeon in microsurgery can be provided for the surgeon.

### Second Embodiment

Hereinafter, a second embodiment of a surgical microscope system, an auxiliary imaging device, and a surgical field imaging method of the present invention will be described.

A surgical microscope system 1 according to the second embodiment is configured similar to the surgical microscope system 1 according to the first embodiment described above except for points to be described below. Thus, according to the surgical microscope system 1 of the second embodiment, effects similar to those of the surgical microscope system 1 according to the first embodiment described above can be acquired except for points described below.

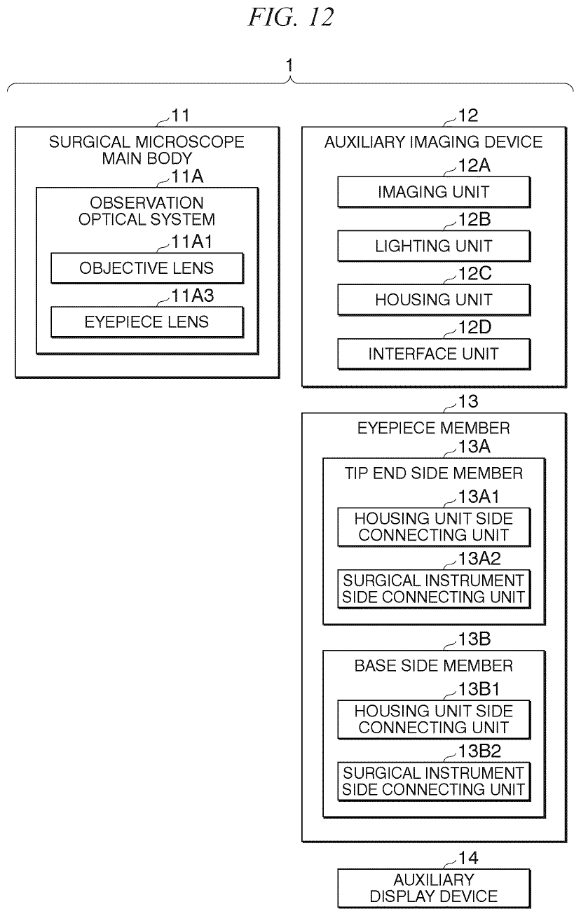

FIG. 12 is a diagram illustrating one example of the surgical microscope system 1 according to the second embodiment.

In the example illustrated in FIG. 12, similar to the surgical microscope system 1 according to the first embodiment, the surgical microscope system 1 according to the second embodiment, for example, is used when microvascular anastomosis is performed in microsurgery.

In the example illustrated in FIG. 12, the surgical microscope system 1 according to the second embodiment includes a surgical microscope main body 11, an auxiliary imaging device 12, a connecting member 13, and an auxiliary display device 14.

The surgical microscope main body 11 according to the second embodiment, for example, is configured to be similar to a surgical microscope described on web sites indicated by the following URLs.

```
  https://topconhealthcare.jp/ja/wp-content/uploads/sites/25/2020/08/OMS-
90_Brochure_E343_Rev1.pdf
  https://www.medicalexpo.com/ja/prod/dre-medical/product-76042-733911.html
```

In the example illustrated in FIG. 12, the surgical microscope main body 11 has an observation optical system 11A. The observation optical system 11A, for example, includes an objective lens 11A1, an eyepiece lens 11A3, and the like. The objective lens 11A1 is disposed to face a surgical field including surgical parts such as two micro blood vessels BL (see FIG. 2) to be anastomosed in microsurgery. The eyepiece lens 11A3 has a function of further enlarging an image that has been enlarged by the objective lens 11A1. A surgeon can check an image of a surgical field through the eyepiece lens 11A3.

In the example illustrated in FIG. 12, a surgeon can check the surgical field through the eyepiece lens 11A3 and also can check surgical parts (two micro blood vessels BL and the like to be anastomosed in microsurgery) with an angle different from that of the surgical microscope main body 11 by using the auxiliary display device 14.

In the surgical microscope system 1 according to the second embodiment, similar to the surgical microscope system 1 according to the first embodiment, the imaging unit 12A of the auxiliary imaging device 12 images a surgical field from which the intima of the inside of the end portion of the micro blood vessel BL to be anastomosed in microsurgery can be checked. For this reason, according to the surgical microscope system 1 of the second embodiment, similar to the surgical microscope system 1 according to the first embodiment, an image of the surgical field illustrated in FIG. 9(B), from which the intima of the inside of the end portion of the micro blood vessel BL to be anastomosed in microsurgery can be checked, seen from the position of the forceps ST1 can be provided for a surgeon.

Furthermore, in the surgical microscope system 1 according to the second embodiment, similar to the surgical microscope system 1 according to the first embodiment, the surgical instrument side connecting unit 13A2 of the tip end side member 13A and the surgical instrument side connecting unit 13B2 of the base side member 13B are connected to the first part ST11, which is brought into contact with the thumb of the surgeon's non-dominant hand, out of the first part ST11 and the second part ST12 of the forceps ST1, In other words, the imaging unit 12A of the auxiliary imaging device 12 is connected to the first part ST11, which is brought into contact with the thumb of the surgeon's non-dominant hand, out of the first part ST11 and the second part ST12 of the forceps ST1 through the connecting member 13.

For this reason, according to the surgical microscope system 1 of the second embodiment, similar to the surgical microscope system 1 according to the first embodiment, when moving the tip end part ST12A of the second part ST12 of the forceps ST1 by moving an index finger to the thumb side, a surgeon can suppress the shaking of the image of the surgical field imaged by the imaging unit 12A.

In addition, in the surgical microscope system 1 according to the second embodiment, similar to the surgical microscope system 1 according to the first embodiment, the auxiliary imaging device 12 images a surgical field with an angle different from that of the surgical microscope main body 11 in the state of being connected to the forceps ST1 held by a surgeon.

For this reason, the surgical microscope system 1 according to the second embodiment, similar to the surgical microscope system 1 according to the first embodiment, can provide a imaged image of the surgical field from the position of forceps ST1 held by a surgeon in microsurgery for the surgeon.

### Third Embodiment

Hereinafter, a third embodiment of a surgical microscope system, an auxiliary imaging device, and a surgical field imaging method of the present invention will be described.

A surgical microscope system 1 according to the third embodiment is configured similar to the surgical microscope system 1 according to the first embodiment described above except for points to be described below. Thus, according to the surgical microscope system 1 of the third embodiment, effects similar to those of the surgical microscope system 1 according to the first embodiment described above can be acquired except for points described below.

In one example of the surgical microscope system 1 according to the third embodiment, the surgical microscope system 1 according to the third embodiment is used when a microsurgical procedure other than microvascular anastomosis (for example, nerve suturing, lymphatic vessel repair, and the like) is performed in microsurgery.

In one example of the surgical microscope system 1 according to the third embodiment, a surgeon can check the surgical field through the eyepiece lens 11A3 and also can check surgical parts (the two nerves, lymphatic vessels, and the like that are anastomosed in microsurgery) with an angle different from that of the surgical microscope main body 11 by using the auxiliary display device 14.

In the surgical microscope system 1 according to the third embodiment, similar to the surgical microscope system 1 according to the first embodiment, the surgical instrument side connecting unit 13A2 of the tip end side member 13A and the surgical instrument side connecting unit 13B2 of the base side member 13B are connected to the first part ST1l, which is brought into contact with the thumb of the surgeon's non-dominant hand, out of the first part ST11 and the second part ST12 of the forceps ST1. In other words, the imaging unit 12A of the auxiliary imaging device 12 is connected to the first part ST11, which is brought into contact with the thumb of the surgeon's non-dominant hand, out of the first part ST11 and the second part ST12 of the forceps ST1 through the connecting member 13.

For this reason, according to the surgical microscope system 1 of the third embodiment, similar to the surgical microscope system 1 according to the first embodiment, when moving the tip end part ST12A of the second part ST12 of the forceps ST1 by moving an index finger to the thumb side, a surgeon can suppress the shaking of the image of the surgical field imaged by the imaging unit 12A.

In addition, in the surgical microscope system 1 according to the third embodiment, similar to the surgical microscope system 1 according to the first embodiment, the auxiliary imaging device 12 images a surgical field with an angle different from that of the surgical microscope main body 1I in the state of being connected to the forceps ST1 held by a surgeon.

For this reason, the surgical microscope system 1 according to the third embodiment, similar to the surgical microscope system 1 according to the first embodiment, can provide a imaged image of the surgical field from the position of forceps ST1 held by a surgeon in microsurgery for the surgeon.

### Fourth Embodiment

Hereinafter, a fourth embodiment of a surgical microscope system, an auxiliary imaging device, and a surgical field imaging method of the present invention will be described.

A surgical microscope system 1 according to the fourth embodiment is configured similar to the surgical microscope system 1 according to the first embodiment described above except for points to be described below. Thus, according to the surgical microscope system 1 of the fourth embodiment, effects similar to those of the surgical microscope system 1 according to the first embodiment described above can be acquired except for points described below.

In one example of the surgical microscope system 1 according to the fourth embodiment, the surgical microscope system 1 according to the fourth embodiment is used when an arbitrary microsurgical procedure (for example, plastic surgery, neurosurgery, orthopedic surgery, ophthalmic surgery, dental surgery, veterinary surgery, and the like) is performed.

In one example of the surgical microscope system 1 according to the fourth embodiment, an imaging unit 12A images surgical parts included in a surgical field imaged by a surgical microscope main body 11 with an angle different from that of the surgical microscope main body 11. For this reason, a surgeon can check the surgical field through an eyepiece unit 11B2 and also can check the surgical parts with an angle different from that of the surgical microscope main body 11 by using an auxiliary display device 14.

In one example of the surgical microscope system 1 according to the fourth embodiment, a connecting member 13 connects a surgical instrument ST (the surgical instrument ST may be a surgical instrument ST other than forceps ST1) held by a surgeon and the auxiliary imaging device 12.

In the surgical microscope system 1 according to the fourth embodiment, the auxiliary imaging device 12 images the surgical field in the state of being connected to a surgical instrument ST held by a surgeon and with an angle different from that of the surgical microscope main body 11.

For this reason, the surgical microscope system 1 according to the fourth embodiment can provide a imaged image of a surgical field from the position of a surgical instrument ST held by a surgeon in a microsurgical procedure (microsurgery) for the surgeon.

### Fifth Embodiment

Hereinafter, a fifth embodiment of a surgical microscope system, an auxiliary imaging device, and a surgical field imaging method of the present invention will be described.

A surgical microscope system 1 according to the fifth embodiment is configured similar to the surgical microscope system 1 according to the first embodiment described above except for points to be described below. Thus, according to the surgical microscope system 1 of the fifth embodiment, effects similar to those of the surgical microscope system 1 according to the first embodiment described above can be acquired except for points described below.

In one example of the surgical microscope system 1 according to the fifth embodiment, the surgical microscope system 1 according to the fifth embodiment is used when an arbitrary microsurgical procedure (for example, plastic surgery, neurosurgery, orthopedic surgery, ophthalmic surgery, dental surgery, veterinary surgery, and the like) is performed.

In one example of the surgical microscope system 1 according to the fifth embodiment, an imaging unit 12A images surgical parts included in a surgical field imaged by a surgical microscope main body 11 with an angle different from that of the surgical microscope main body 11. For this reason, a surgeon can check the surgical field through an eyepiece unit 11B2 and also can check the surgical parts with an angle different from that of the surgical microscope main body 11 by using an auxiliary display device 14.

In one example of the surgical microscope system 1 according to the fifth embodiment, a connecting member 13 connects a surgical instrument ST (for example, a hook and the like) held by a surgical assistant and the auxiliary imaging device 12.

In the surgical microscope system 1 according to the fifth embodiment, the auxiliary imaging device 12 images the surgical field in the state of being connected to a surgical instrument ST held by a surgical assistant and with an angle different from that of the surgical microscope main body 11.

For this reason, the surgical microscope system 1 according to the fifth embodiment can provide a imaged image of a surgical field from the position of a surgical instrument ST held by a surgical assistant in a microsurgical procedure (microsurgery) for a surgeon.

### <Application Example of Surgical Microscope System 1 According to First to Fifth Embodiments>

By providing an image of a surgical field imaged by the surgical microscope main body 11 and an image of a surgical field imaged by the imaging unit 12A for a surgical support robot, a safe robot-supported surgery procedure in which a surgical support robot appropriately operates can be realized.

By applying an image of a surgical field imaged by the surgical microscope main body 11 and an image of a surgical field imaged by the imaging unit 12A to training of microvascular anastomosis, practices and instructions for microvascular anastomosis can be easily performed.

Although the embodiments of the present invention have been described in detail with reference to the drawings as above, a specific configuration is not limited to such embodiments, and changes can be appropriately made in a range not departing from the concept of the present invention. The components described in each of the embodiments and each of the examples described above may be combined together.

### REFERENCE SIGNS LIST

1 Surgical microscope system
11 Surgical microscope main body
11A Observation optical system
11A1 Objective lens
11A2 Imaging element
11B Display device
11B1 Image presenting unit
11B2 Eyepiece unit
11B3 Display
11A Observation optical system
11A1 Objective lens
11A3 Eyepiece lens
12 Auxiliary imaging device
12A Imaging unit
12B Lighting unit
12C Housing unit
12D Interface unit
14 Auxiliary display device
13 Connecting member
13A Tip end side member
13A1 Housing unit side connecting unit
13A2 Surgical instrument side connecting unit
13B Base side member
13B1 Housing unit side connecting unit
13112 Surgical instrument side connecting unit
ST Surgical instrument
ST1 Forceps
ST11 First part
ST11A Tip end part
ST11B Base part
ST11C holding part
ST11D First attachment part
ST11E Second attachment part
ST12 Second part
ST12A Tip end part
ST12B Base part
ST12C Holding part
ST2 Needle holder
CP Clip
SR Suture thread
ND Suture needle
BL Micro blood vessel

1. A surgical microscope system comprising:
a surgical microscope main body having an observation optical system including at least an objective lens; and
an auxiliary imaging device including at least an imaging unit and a lighting unit,
wherein the auxiliary imaging device images a surgical field in the state of being connected to a surgical instrument held by a surgeon or a surgical assistant and with an angle different from that of the surgical microscope main body.

2. The surgical microscope system according to claim 1,
wherein forceps are included in the surgical instrument,
the surgical microscope system includes a connecting member connecting the forceps and the auxiliary imaging device to each other, and
the imaging unit of the auxiliary imaging device images a surgical field from which an intima of the inside of a blood vessel to be anatomosed in microsurgery can be checked.

3. The surgical microscope system according to claim 2,
wherein the auxiliary imaging device has a housing unit housing the imaging unit and the lighting unit,
the connecting member includes a tip end side member disposed on a tip end side of the forceps and a base side member disposed on a base side of the forceps relative to the tip end side member,
the tip end side member includes a housing unit side connecting unit connected to the housing unit and a surgical instrument side connecting unit connected to the forceps, and
the base side member includes a housing unit side connecting unit connected to the housing unit and a surgical instrument side connecting unit connected to the forceps.

4. The surgical microscope system according to claim 3,
wherein the forceps include a first part brought into contact with a thumb of a surgeon and a second part brought into contact with an index finger of the surgeon, and
wherein the surgical instrument side connecting unit of the tip end side member and the surgical instrument side connecting unit of the base side member are connected to the first part.

5. An auxiliary imaging device included in a surgical microscope system including a surgical microscope main body having an observation optical system including at least an objective lens, the auxiliary imaging device comprising:
at least an imaging unit and a lighting unit;
wherein a surgical field is imaged in the state of being connected to a surgical instrument held by a surgeon or a surgical assistant and with an angle different from that of the surgical microscope main body.

6. A surgical field imaging method comprising:
a first imaging step of imaging a surgical field by using a surgical microscope main body having an observation optical system including at least an objective lens; and
a second imaging step of imaging the surgical field with an angle different from that of the surgical microscope main body by using an auxiliary imaging device having at least an imaging unit and a lighting unit,
wherein the auxiliary imaging device images the surgical field in the state of being connected to a surgical instrument held by a surgeon or a surgical assistant.
